(19) United States
(12) Patent Application Publication
(10) Pub. No.: US 2004/0248871 A1
(43) Pub. Date: Dec. 9, 2004

(54) Use of lysyl oxidase inhibitors for cell culture and tissue engineering

(76) Inventors: Jean Farjanel, Lyon (FR); Odile Damour born Baudoux, Laval (FR); Pascal Sommer, Laval (FR); Fabienne Raye, Pierre De Chandieu (FR); Michel-Henri Fessy, Millery (FR)

Correspondence Address: YOUNG & THOMPSON, 745 SOUTH 23RD STREET, 2ND FLOOR, ARLINGTON, VA, 22202, US

(21) Appl. No.: 10/485,542

(22) PCT Filed: Aug. 2, 2002
(86) PCT No.: PCT/FR02/02789

(30) Foreign Application Priority Data
Aug. 3, 2001 (FR) 0110443

(51) Int. Cl. A61K 31/33; A61K 31/275; A61K 31/198; A61K 31/13; A61K 31/15; A61K 39/395

(52) U.S. Cl. 514/183; 514/562; 514/522; 514/664; 514/673; 424/146.1

(57) ABSTRACT

A subject of the invention is the use of lysyl oxidase inhibitors in the context of the implementation of in vitro cell culture methods which are capable of being used in tissue therapy, or cell therapy, or in experimental pharmacology.

A subject of the invention is the use of lysyl oxidase inhibitors in the implementation of in vitro cell culture methods capable of being used in tissue or cell therapy, or in experimental pharmacology.

The lysyl oxidases are amine oxidases which induce the cross-linking of collagens and elastin while catalyzing and which catalyze the oxidative deamination of lysyl and hydroxylysyl residues to corresponding α-aminoadipic-S-semialdehyde (see journal article, Smith-Mango & Kagan, Matrix Biology 1998, 16: 387): RCH₂NH₂+O₂+H₂ O→RCHO+NH₃+H₂O₂. The aldehyde residues formed will be subjected to a series of spontaneous condensations with other neighbouring non-modified aldehyde or amine functions, leading to intra and inter-molecular bonds. These condensations are at the origin of the intra and intermolecular bridgings of the collagens and elastin. Among the direct LO activity inhibitors, which act on the active site of the enzyme, βAPN is the most commonly used (see journal article by H. M. Kagan Acta Tropica 77 (2000) 147-152).

The cross-linking dependent on the LOs of the collagens and elastin is an essential parameter of the formation of tissues and implants which are neosynthesized in vitro: The absence of cross-linking does not allow the constitution of resistant tissues, too much cross-linking (or an inappropriate chemical cross-linking) lead to too great a rigidity and a retractation of the tissues.

The present invention follows from the Inventors demonstrating the fact that the addition of an LO inhibitor specific to chondrocytes, cartilage-specific cells, allows the cancelling out of the dedifferentiation of these cells. In fact, the major problem with the formation of cartilage implants by the chondrocytes is that the latter dedifferentiate, and lose their potential to produce collagen cartilage (collagens of cartilage type: II, IX, XI and X). Without this inhibitor, the dedifferentiated chondrocytes synthesize types of abnormal collagens in the healthy cartilage (I, III). The temporary addition of the LO inhibitor therefore has an important and reversible effect on the formation of the cartilage, the specific physical properties of which are linked to the presence of the collagens which constitute it.

Moreover, the Inventors have also demonstrated that the addition of the same LO inhibitor during the constitution of a model of reconstructed skin also translates into an effect on the phenotype of the cells. The cell components of the tested model of reconstructed skin are fibroblasts and keratinocytes. The fibroblasts synthesize in particular the fibrillar collagens (type I and III) and the elastin of the dermis, while the keratinocytes differentiate to form all the layers of the epidermis as far as the stratum corneum which has a barrier function. In the presence of the LO inhibitor, the properties of the reconstructed skin are modified. It is observed that the absence of cross-linking of the collagens allows a better colonization of the support sponge by the fibroblasts and an increase in formation of the extracellular matrix, as well as in vitro reduction of the retraction of the reconstructed skin. On the other hand, a better organization of the epidermis is observed, with a very well formed granular layer. The temporary addition of LO inhibitor therefore has a positive effect on control of the formation and organization of the reconstructed skin while facilitating the preparation of this material in large quantities.

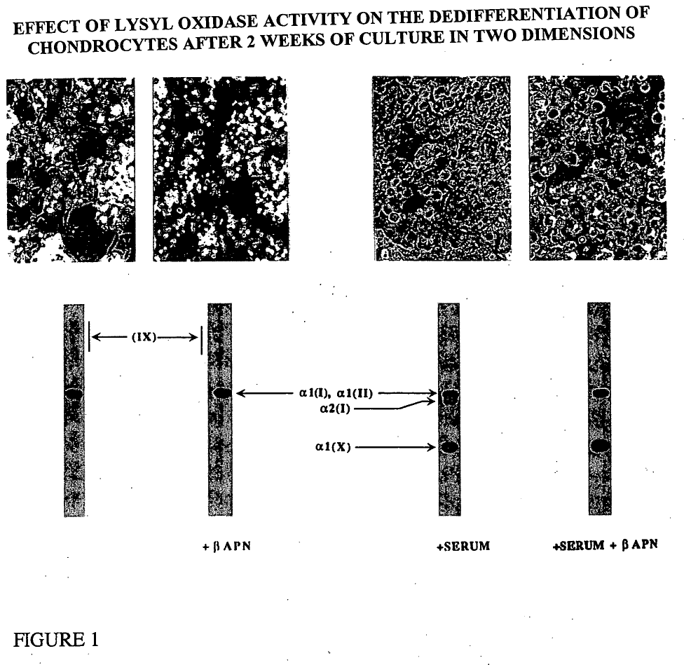

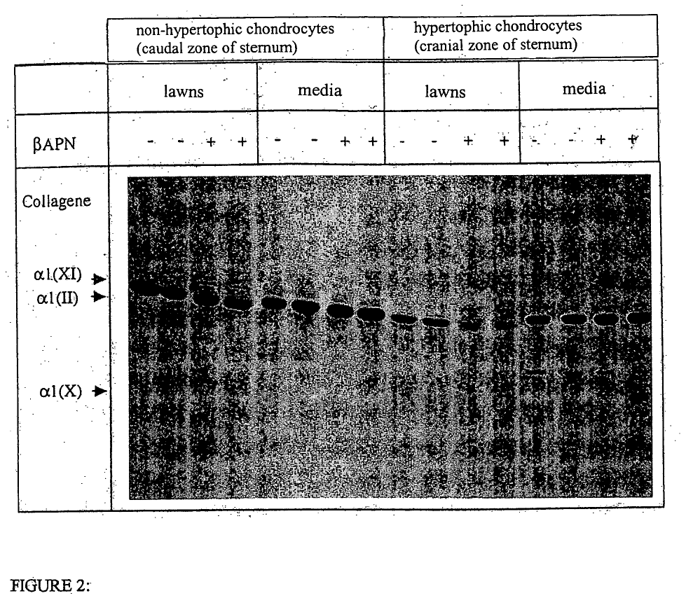

Starting with cartilage biopsies, and in the presence of LO inhibitors, the Inventors have demonstrated that it becomes possible to multiply chondrocytes on a two-dimensional plastic support in the presence of serum from the donor according to standard conditions, while obtaining the production of an extracellular matrix which is stripped of any abnormal collagen, even after 2 weeks of culture, and despite an increased solubilization of the collagens synthesized in the culture medium (FIGS. 1, 2). Such a matrix sees its fluidity reduce during the culture period: the optimal culture period is chosen as a function of the quantity of matrix produced and its desired fluidity. It is then unnecessary to use exogenous support the rejection or any defective resorption of which would cancel out the favourable properties which have already been obtained.

In the absence of typical LO activity inhibitor, βAPN, the extracellular matrix is very lacking in cartilage-type collagens, and mainly constituted by type I collagens as well as, to a smaller extent, by type III collagen, i.e. from collagens which do not participate in the development of the cartilage. Moreover, the matrix has an absence of fluidity which is incompatible with any recourse to injections by syringe.

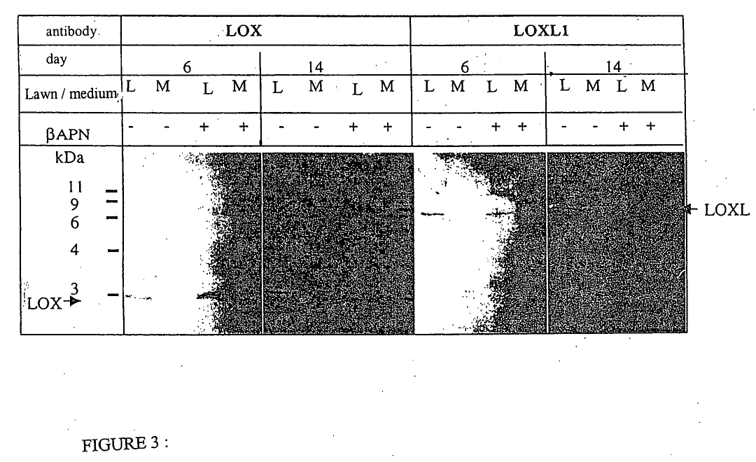

The expression of several isoforms of Lysyl oxidase (at least LOX and LOL1) by the chondrocytes is evident, but it is not modified by treatment with βAPN (FIG. 3) which only inhibits the activity of the Enzyme (Table I).

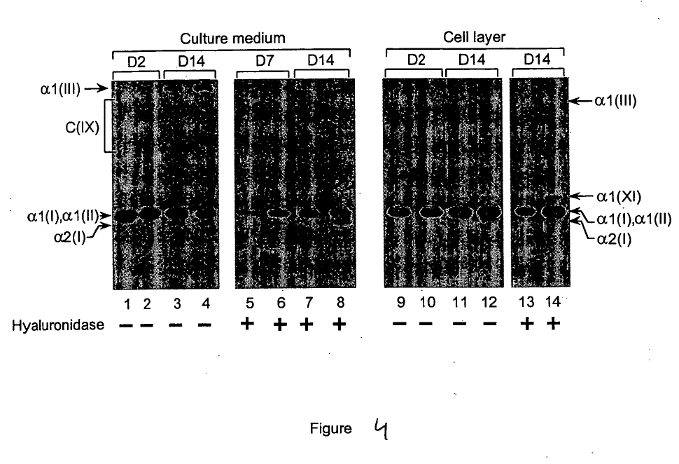

The addition of βAPN, used at different concentrations (between 50 or 200 μg/ml), allows the formation of reconstructed human skins the retraction of which can be reduced in vitro. Moreover, βAPN at 50 μg/ml improves the colonization of the sponge by the fibroblasts and the extension of the synthesis of the extracellular matrix. It also improves the organization of the epidermis, with a very well formed granular layer which generates a highly structured stratum corneum. This effect on the differentiation of the epidermis and the reduction of retraction reduces the formation of flakes which detach themselves and can facilitate the preparation of reconstructed skin (FIG. 4).

The use of LO activity inhibitors can be applied to any cell culture in order to obtain any tissue implant, since the collagens and the LOs are components and elements which are present in all animal tissues. The culture conditions can vary however as a function of the cell types used and substrates targeted.

A subject of the invention is the use of direct or indirect lysyl oxidase (LO) inhibitors, as inhibitors of the dedifferentiation of cells in culture in vitro, in order to keep practically constant the phenotype of said cells in the context of the implementation of methods of in vitro culture of the latter, for almost the whole of the culture period.

A more particular subject of the invention is therefore the use of above-mentioned inhibitors for the preparation of differentiated cells the phenotype of which is preserved.

The invention still more particularly relates to the use of above-mentioned inhibitors for the preparation of a cell matrix, constituted by differentiated cells cultured in the presence of said inhibitors, and from the extracellular medium conditioned by said cells and in contact with said matrix, said cell matrix being able to be used as tissues or tissue implants.

A subject of the invention is also the above-mentioned use of direct LO inhibitors chosen from:

A) the primary amines reacting with the carbonyl group of the active site of the LOs, and more particularly those which produce, after binding with the carbonyl, a product stabilized by resonance, such as the following primary amines:

ethylenediamine,

hydrazine, phenylhydrazine, and their derivatives, semicarbazide, and urea derivatives,

aminonitriles, such as β-aminopropionitrile (β-APN), or 2-nitroethylamine,

unsaturated or saturated haloamines, such as 2-bromo-ethylamine, 2-chloroethylamine, 2-trifluoroethylamine, 3-bromopropylamine, p-halobenzylamines,

selenohomocysteine lactone,

B) copper chelating agents, penetrating or not penetrating the cells in culture,

C) the anti-LO blocking antibodies, directed against the active site of the LOs.

A subject of the invention is also the above-mentioned use of indirect LO inhibitors chosen from:

A) the compounds blocking the aldehyde derivatives originating from the oxidative deamination of the lysyl and hydroxylysyl residues by the LOs, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphinate, 2-acetamidoethyl-2-acetamidoethanethiol sulphanate, sodium-4-mercaptobutanesulphinate trihydrate.

B) the compounds inhibiting the biosynthesis of LOs such as the antisenses.

A more particular subject of the invention is the above-mentioned use of LO inhibitors chosen from β-APN and/or D-penicillamine.

The invention also relates to the use of LO inhibitors as defined above, for the implementation of methods of in vitro culture of all cells of human or animal origin, which said cells are maintained in a phenotype with constant differentiation, and are chosen in particular from chondrocytes of the cartilages, cornea cells, skin cells (such as dermal fibroblasts, and epidermal keratinocytes), endothelial cells of the vessels, bone osteoblasts, hepatocytes, renal cells, muscle cells, stem or pluripotent cells.

A subject of the invention is also the use of LO inhibitors as defined above, for the implementation of in vitro cell culture methods, in order to obtain cells capable of being used in cell therapy, or in experimental pharmacology, in particular in the screening of medicaments.

The invention also relates to the use of LO inhibitors as defined above, for the implementation of in vitro cell culture methods in order to obtain tissues, such as skin or cartilage tissues, said tissues being capable of being used as grafts or implants in tissue therapy, or in experimental pharmacology, in particular in the screening of medicaments.

A subject of the invention is also any cell culture medium characterized in that it contains one or more LO inhibitors as defined above.

The invention also relates to an in vitro cell culture method as defined above, during which the phenotype of said cells is maintained at a stage identical to that in which said cells were found initially during their culture, said method comprising the culture of said cells in an appropriate medium containing one or more LO inhibitors as defined above.

A more particular subject of the invention is a method for constantly maintaining the phenotype of differentiated cells in culture at a stage which is close or identical to that in which said cells were found during their culture, characterized in that it comprises the culture of said cells in an appropriate medium containing one or more LO inhibitors as defined above.

A subject of the invention is also a method for preparing differentiated cells with an identical phenotype, or cell implants constituted by such cells, characterized in that it comprises:

the implementation of an in vitro cell culture method as defined above,

if appropriate, one or more stages of washing the cells in culture using an appropriate buffer solution, in order to completely or partially eliminate the LO inhibitor or inhibitors used,

if appropriate, a stage of enzymatic digestion of the extracellular material capable of being formed, using appropriate enzymes,

if appropriate, a stage of recovery of the cells cultured.

The invention also relates to a method for preparing in vitro a cell matrix as defined above, capable of being used as tissues or tissue implants, constituted by differentiated cells, characterized in that it comprises:

the implementation of an in vitro cell culture method as defined above, until formation of a cell matrix as defined above, which is sufficient to constitute a tissue stroma,

if appropriate, one or more stages of washing of the cell matrix in culture using an appropriate buffer solution, in order to completely or partially eliminate the LO inhibitor or inhibitors used,

if appropriate, a stage of recovery of the cell matrix as defined above.

More particularly a subject of the invention is a method for preparing in vitro tissues or tissue implants as defined above, used for the neosynthesis of cartilage implants when said method is carried out from chondrocytes, or for the preparation of cutaneous substitutes when said method is carried out from fibroblasts and/or keratinocytes.

The invention also relates to a method for screening molecules of pharmacological interest, in particular medicaments, characterized in that it comprises:

the implementation of an in vitro cell culture method as defined above, if appropriate, until the formation of a cell matrix which is sufficient to constitute a tissue stroma,

if appropriate, one or more stages of washing the cell matrix in culture using an appropriate buffer solution, in order to completely or partially eliminate the LO inhibitor or inhibitors used,

a stage of placing the molecule tested in the presence of the cells or tissues obtained during the preceding stages,

the detection of any effect of said molecule on the above-mentioned cells or tissues.

A subject of the invention is also the differentiated cells with the phenotype maintained, or cell implants constituted by such cells, as defined above, characterized in that they contain LO inhibitors as defined above, if appropriate, as traces.

The invention also relates to the cell matrices or tissues or tissue implants based on cells as defined above, characterized in that they contain LO inhibitors as defined above, if appropriate, as traces.

A more particular subject of the invention is the above-mentioned cells or cell implants, or cell matrices or tissues or tissue implants, as obtained by implementation of a method as defined above.

The invention also relates to the cartilage implants comprising an extracellular matrix essentially free from collagens which are normally absent from healthy cartilage (namely collagens I and III), and comprising all the specific collagens of cartilage (namely collagens II, IX, XI, and if appropriate X).

A more particular subject of the invention is the above-mentioned cartilage implants, characterized in that they contain LO inhibitors as defined above, if appropriate, as traces.

A more particular subject of the invention is also the above-mentioned cartilage implants, as obtained by implementation of a method as defined above.

The invention also relates to implants of chondrocytes, the initial phenotype of which, of cartilage type, was maintained during the preliminary phase of cell multiplication in vitro.

A subject of the invention is also the above-mentioned tissue implants corresponding to implants of reconstituted skin, or cutaneous substitutes, as obtained by implementation of a method according to claim 11 carried out from fibroblasts and/or keratinocytes.

The invention is further explained by the description which follows of the possible uses of LO inhibitors.

The production of tissue implants in Tissue Engineering in general must overcome a major obstacle: avoiding producing an abnormal tissue having physico-chemical properties which are incompatible with the envisaged use. The culture of the cells in vitro in order to multiply them and to produce an extracellular matrix unfortunately brings with it an alteration of their biochemical phenotype which is more or less profound according to the type of cell culture.

The culture of chondrocytes until now has, from this point of view, always been accompanied by this phenotypic alteration. The solutions proposed until now for the cultures of chondrocytes have consisted of adding to the culture (or sometimes removing from it) soluble factors (hormones, vitamins, essentially cellular factors), whether this is carried out on a plane plastic support or in a three-dimensional support of various kinds (more or less cross-linked collagen sponges combined with various components, ceramics etc.).

The production of reconstructed skin is based on the interaction of fibroblasts and keratinocytes placed under particular conditions. The interventions allowing improvement of this production are limited and badly defined, often on an empirical basis. Several nutrients thus prove to be indispensable and have been added to the culture conditions. The treatment with βAPN has the peculiarity of being directed at the two cell types.

The method of the present invention applies to any type of culture in Tissue Engineering. It consists of acting on the physical state of the extracellular matrix synthesized by the cells in culture in order to modulate the signals that this sends to the cells, via various cell receptors, and thus to regulate the state of differentiation of the isolated cells from biopsies.

LO inhibition (in particular by βAPN) has until now been used in methods for producing collagens, for inhibiting the cross-linking of the collagens secreted by the cells and thus improving the conditions of their solubilization. Such an inhibitor is, for this reason, recognized in standard fashion as altering the extracellular matrix and until now has been used in only three circumstances:

the production of non-cross-linked, easily extractable collagens,

the fundamental study of the action mechanism of the LO,

to contribute to inhibiting the excessive bridging of collagens in certain pathologies causing fibrosis.

Outside of these three fields, the inhibition of the LO activity has until now appeared to be an obstacle, not an advantage.

The principal advantages of the present invention are the following.

βAPN, the typical LO activity inhibitor, is an amino-nitrile. This compound, which is very inexpensive, has until now never been used to maintain the phenotype of the cells in culture. Non-toxic at the doses used for the cells in culture, its action can nevertheless reach other cells within an organism: for this reason it cannot be used without risks for therapeutic purposes, which necessarily have a target. Its elimination from the cultures before their use as implants is aided by successive washings to deplete the βAPN in the culture. The other specific LO activity inhibitors could usefully be compared with regard to their level of tolerance in vivo in such a model.

The effectiveness of βAPN at the standard concentration of 50 μg/ml was total after 2 weeks of culture where, in its absence, the dedifferentiation of the chondrocytes had become maximal. This concentration is useful for the treatment of reconstructed skins.

The stopping of treatment by βAPN then allows restoration of the activity of the LOs which are continuously secreted (or even expressed more in the presence of βAPN) by the cells and have a limited activity period. Although the inhibition of the LOs by βAPN is irreversible, the elimination of βAPN, by washings and stopping the treatment, allows the restoration in vitro of the activity of this enzyme at the chosen moment. The desired cross-linking of the collagen and elastin fibrils, substrates of the LOs, can thus be programmed.

The method of the present invention applies to any type of culture in Tissue Engineering, i.e.:

any type of support: the most simple such as the plastic support of the culture dishes, or the most sophisticated such as certain three-dimensional culture models.

any type of cells for various tissue implants (cartilage, bone, skin, cornea, blood vessels, etc). The treatment described can be used for two types of implants:

a) suspension of chondrocytes multiplied in vitro. Moreover, the treatment of the culture beforehand with βAPN improves the isolation yield of the viable cells by trypsination.

b) cartilaginous matrix rich in chondrocytes and with defined viscosity.

In the case of articular cartilages locally damaged or necrotized (in particular the hip), it can be desirable to have recourse to injections of a matrix rich in non-dedifferentiated chondrocytes. The fluidity of the cell lawn is a function of the duration of the culture and can therefore be easily chosen by the surgeon according to requirements. This advantage is of course common to any other situation encountered in Tissue Engineering.

The cell lawns thus obtained after a given period of contact with an LO inhibitor can also serve as support for study in pharmacotoxicology.

### 1. DETAILED DESCRIPTION

1.1.) Material and Methods

Chondrocyte Culture Model:

a) The experimental chondrocyte culture model is the one with the culture of chicken embryo chondrocytes on plastic in two dimensions. The embryonic state of the cells allows a high level of synthesis of the collagens, unlike the adult chondrocytes. The chicken sternum from which the chondrocytes originates Makes it possible to obtain two pure sub-populations of chondrocytes capable or not capable of subsequently hypertrophying (respectively, the chondrocytes of the cranial or caudal section of the sternum). The biopsies of bovine or human cartilage do not permit this distinction. The results which are described always distinguish the sub-population of the chondrocytes studied.

The cultures with 4 mM L-glutamine antibiotics, and 25 μg/ml ascorbate (factor necessary for the stability of the secreted collagen triple helix units) are continued for 2 weeks in the presence or absence of 10% serum, optionally replaced by 100 ng/ml insulin, and by 50 μg/ml βAPN. The absence of serum necessitates the addition of protective agents (1 mM cysteine and 1 mM pyruvate). A day before taking the samples, 10 μCi of 14C Proline is added to the cultures the lawns or media of which are then pepsinated (0.2 mg pepsin/ml/pH 4/24 hours) and subjected to electrophoretic (SDS PAGE gradient of 4.5 at 15% acrylamide) then fluorographic analysis. The fluorographs then reveal the only pepsin-resistant protein bands, essentially the collagens and lipocalines (proteins with lipid transport). The known position of these bands allows the indication of the dedifferentiation of the cells by the appearance of the α2 I chain of collagen I associated with the lowering of the synthesis of the collagens of cartilage type.

The Lysyl oxidase activity was assayed with 300,000 dpm of labelled elastin on the Lysine, substrate of the enzyme (Shackelton and Hulmes Anal. Biochem. (1990) 185: 359-362). The determination of the presence of Lysyl oxidases in the cultures of chondrocytes is carried out according to the standard methods of immunodecoration of the supports onto which the bands separated from the proteins extracted from the corresponding culture media or from the cell lawns have been transferred.

b) The cultures of normal human chondrocytes originate from cartilages taken during hip prosthesis or ligamentoplasty surgery, then subjected to enzymatic digestion. These cartilage biopsies are considered as res nullus.

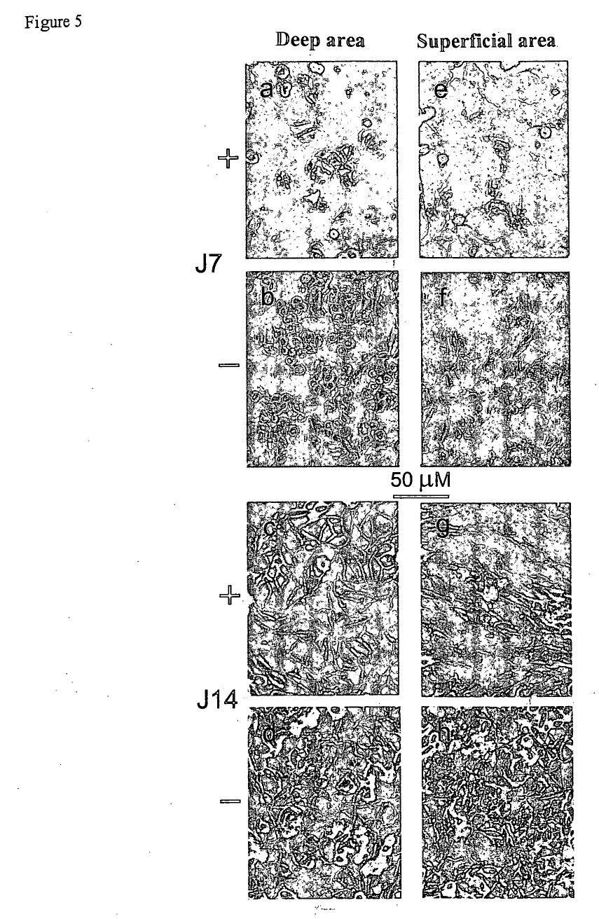

The chondrocytes extracted from the cotyloid ligament have been used for the two-dimensional culture on plastic in the presence of BAPN: the cultures obtained after enzymatic digestion of the cartilages in the presence or absence of hyaluronidase were compared (FIGS. 4 and 5).

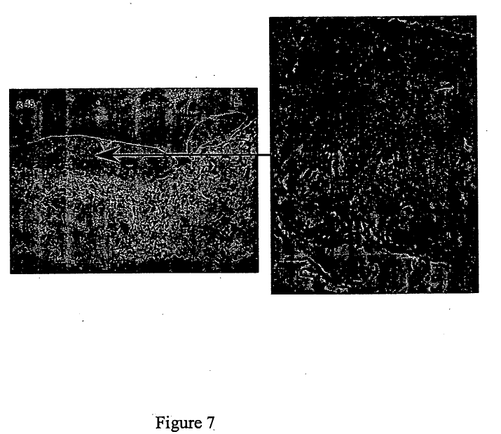

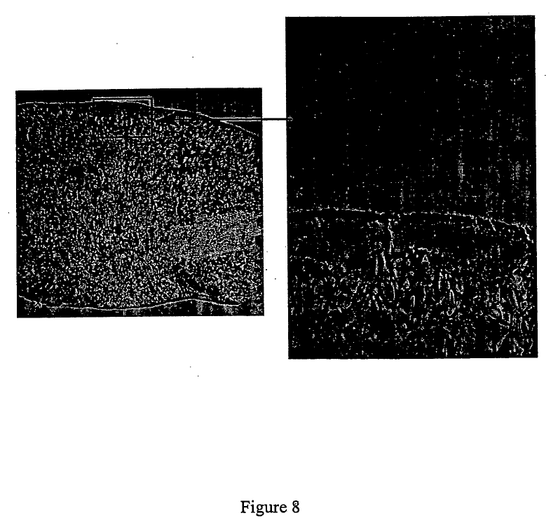

The chondrocytes obtained after ligamentoplasty have been used for the three-dimensional culture in collagen-GAG-chitosan sponge (Collombel et al. 1987)* in the presence or absence of BAPN (FIGS. 7 and 8). * Collombel C., Damour O., Gagneu C., Poinsignon F., Echinard C., Marichy J.: Peau artificielle et son procédé de fabrication. French Patent No. 87-08752 (15th Jun. 1987), European Patent No. 88-420194.8 (14th Jun. 1988).

Reconstructed Skin Culture Model:

The method allowing the formation of the reconstructed skin is that described in the French Patent 87-08252 of the 15th Jun. 1987.

The support of the reconstructed skin model is constituted by a dermal substrate (DS) with a base of collagen-glycosaminoglycan cross-linked by chitosan according to the technique published by Duplan-Perrat et al (J Invest Dermatol 2000 114:365). Fibroblasts are inoculated the inside of the DS in order to obtain an equivalent dermis. The reconstituted skin (RS) is obtained by inoculating the keratinocytes after 15 days (day 15) of culture in equivalent dermis. The dermal fibroblasts are obtained from explants of human prepuces after dermal-epidermal separation and cultured in monolayer in DMEM medium (Sigma) supplemented by 10% fœtal calf serum, 4 mM L-glutamine, EGF (Austral Biologic) at 10 ng/ml, penicillin at 100 UI/ml, gentamicin at 100 μg/ml and amphotericin B at 1 μg/ml. The keratinocytes are from a human skin biopsy sample after dermal-epidermal separation by trypsination. The cells are inoculated nutritive layers (irradiated fibroblasts) and cultured in DMEM and HAM F12 at 30% supplemented with fœetal calf serum at 10%, EGF at 10 ng/ml, hydrocortisone at 1.6 ng/ml, umulin at 0.12 UI/ml, choleratoxin at 0.1 nM, triiodothyronine at 0.2 μM, adenine at 24 μg/ml, 4 mM L-Glutamine, penicillin at 100 UI/ml, gentamicin at 100 μg/ml and amphotericin B at 1 μg/ml.

A mixture with a base of bovine collagen of type I and III (72%), glycosaminoglycan (8%) cross-linked by chitosan (20%) is poured into 6-well plates. The dermal substrates thus obtained are then frozen at −80° C. and lyophilized. The fibroblasts are inoculated in the dermal substrate, rehydrated and sterilized at a rate of 250,000 cells per cm². Keratinocytes are inoculated after 15 days (day 15) at a density of 200,000 to 250,000 cells/cm². The reconstructed skins are cultured for 8 days in immersion, in the medium described previously supplemented with 50 μg/ml of vitamin C. Then the reconstructed skins are raised to the air-liquid interface using a simplified culture medium with a DMEM base, supplemented with fœtal calf serum, EGF, hydrocortisone, umulin, L-glutamine, penicillin, gentamicin, and amphotericin B, and ascorbic acid, at the concentrations described above. The reconstructed skins are cultured up to day 60. Samples are taken at day 30 and day 60 for the different analyses.

Use of the Lysyl Oxidase Inhibitor:

βAPN (50 μg/ml) is added immediately upon inoculation, to the culture medium which is renewed every 2 days and applied to the cultures of chondrocytes as well as to those of fibroblasts then of keratinocytes during the formation of the reconstructed skin. The concentration of 50 μg/ml is not toxic for the cells and does not appreciably affect the cell multiplication. Higher concentrations of 200 μg/ml prove to have little effect on the viability and the multiplication of the fibroblasts and the keratinocytes in isolated cultures.

1.2.) Results

Antibodies specific to various enzymes of the lysyl oxidase family have for the first time allowed identification of these different isoforms in the chondrocytes, by immunodecoration after electrophoretic separation as well as by immunomarking of cell lawns observed through a fluorescence microscope. For the first time, a specific lysyl oxidase activity was also demonstrated in the culture media of the chondrocytes (Table I).

The anti LO antibodies have also allowed detection of these isoforms in the reconstructed skin. Some results have shown a strong expression of the LOXs (FIG. 4), LOXL1 and LOXL2 at the level of the dermis, epidermis and dermoepidermal junction. The observation of an expression of these isoforms of LO in keratinocytes is completely original at this level, where there is no synthesis either of collagen or of elastin.

Regulation of the “Cartilage” Phenotype of Chondrocytes:

In the presence of serum, with which the rates of multiplication and protein synthesis are optimal, the chondrocytes studied clearly dedifferentiate after the first week of culture, which is in accordance with the data in the literature. The addition of βAPN throughout the culture eliminates all dedifferentiation, as the absence of the collagen α2I chain on the fluorographs attests. Similarly, βAPN also eliminates the morphological alterations which are moreover observed in the first days in the non-treated cultures where the cells of fibroblastic form are easily seen.

In the absence of serum, the chondrocytes adhere well on the plastic support but spread with great difficulty, or even not at all in the case of the caudal chondrocytes. The presence of an anabolic agent of the chondrocytes, such as insulin, somewhat increases the rates of synthesis, does not induce biochemical dedifferentiation with or without βAPN, but shows a morphological alteration in the absence of βAPN (FIG. 1).

Thus the presence of serum in the culture medium is nevertheless desirable in order to obtain a high rate of multiplication and of syntheses, βAPN allowing the elimination of the alteration of the biochemical and morphological phenotype.

The cultures of chondrocytes thus treated but studied between the 2nd and the 3rd day have shown the presence of normal collagens in the cell lawn as well as in the culture medium. Nevertheless, βAPN has allowed the normal appearance of collagen X by the hypertrophic chondrocytes: the lowering of this synthesis, in the absence of βAPN, is known to precede the synthesis of collagen I, dedifferentiation marker (FIG. 2). These collagens, all of cartilage type, therefore do seem to acquire, under the effect of bridgings dependent on LO, a structure which would progressively contribute to the sending of signals to the chondrocytes, which is at the origin of the alteration of the biochemical phenotype in culture in vitro.

The origin of this inhibition of dedifferentiation by the βAPN remained to be proven. The cultures in the presence of βAPN do not at all modify the synthesis and the secretion of these LOs (FIG. 3). βAPN therefore does act only on the amine oxidase activity of the enzyme, and not on its secretion.

Application of the Model to Human Cartilage Cells.

The method of extraction of the cells by enzymatic digestion of the cartilage allows different sub-populations of chondrocytes within a single biopsy of cartilage to be demonstrated. This is the case of the superficial and deep chondrocytes of the cotyloid ligament of the femoral cartilage after addition of hyaluronidase to the extraction medium. On inoculation, these two populations of cells do produce collagen II, but not collagen I. Only the deep chondrocytes will then be capable, in the presence of βAPN of preserving their morphology and their phenotype for the following two weeks when they multiply producing an extracellular matrix of cartilaginous type, without collagen I.

It is therefore necessary to use for the purposes of Tissue Engineering areas of cartilage the populations of chondrocytes sensitive to βAPN of which have been correctly isolated.

Nevertheless, human chondrocytes dedifferentiated in terms of the period of culture necessary for their multiplication in culture on plastic, can advantageously be placed in three-dimensional (3D) culture in the presence of βAPN. The placing in 3D culture not being sufficient, in itself, to ensure a return to the cartilage phenotype lost during the 2-dimensional culture in the presence of serum, as was described for 3D cultures in agarose. Effectively, the triple label immunofluorescence of the collagen I, collagen II and aggrecan on fractions obtained from three-dimensional cultures of chondrocytes in sponge has allowed the favourable effect of BAPN on the preservation of the cartilage phenotype of the chondrocytes after three months of culture to be demonstrated:

an extension of the area of deposition of collagen II in the sponge and a clear increase in the intensity of marking of the collagen II and of the aggrecan (cartilage markers) combined with a strong decrease in that of the collagen I due to the βAPN.

It is necessary to ensure the effectiveness of the treatment intended to eliminate βAPN from the cell culture of before its use as an autologous tissue implant. This operation meets two requirements:

1—restoring a normal Lysyl oxidase activity within the implanted cartilage which is being repaired.

2—eliminating the risk, hypothetical but not demonstrated, of a possible harmful effect of βAPN on the cells of the patient.

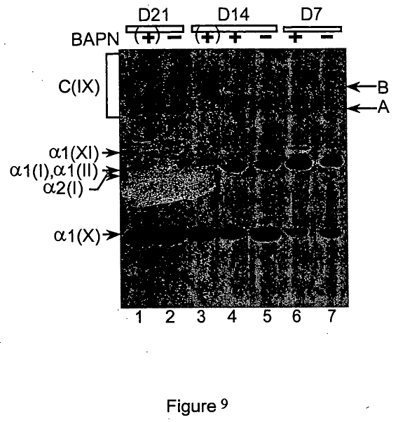

The modifications, observed for the first time, under the effect of the inhibition by βAPN of the Lysyl oxidase activity, in the bridgings between chains of collagen IX proved to be very precocious (preceding the appearance of collagen I dedifferentiation marker) and reversible (at the latest within the 6 days which follow the elimination of βAPN) (FIG. 9). We have thus proved that repeated washings of the cell lawn by PBS preceding the use of culture medium without βAPN prove to be sufficient to eliminate all trace of active βAPN.

Moreover it has been verified that high density inoculation does slow the appearance of the dedifferentiation in 2D culture.

Regulation of the Formation of the Reconstructed Skin:

Preliminary Study of βAPN on the Fibroblasts and the Keratinocytes Cultured in Monolayer

The cell count shows that βAPN only has an inhibiting effect on the proliferation of fibroblasts and keratinocytes in relatively strong doses. In fact, the number of these cells is reduced by approximately 50% compared to the control when they are cultured in the presence of 600 μg/ml of βAPN. However, at the concentration of 200 μg/ml, the proliferation of the fibroblasts is slightly increased, while that of the keratinocytes is reduced by approximately 10%. On the other hand, no toxic effect of βAPN is observed on the fibroblasts and the keratinocytes at confluence up to 800 μg/ml. The results of the MTT viability test confirm those of the cell count. In conclusion, these results indicate that βAPN, at high concentrations (greater than 500 μg/ml), has an inhibiting effect on the proliferation of fibroblasts and keratinocytes. It must be emphasized that βAPN at 200 μg/ml can optionally cause an increase in cell activity (proliferation and viability) on the fibroblasts inoculated at a low density, and on the fibroblasts and the keratinocytes inoculated at a high density.

Reconstructed Skins (RS)

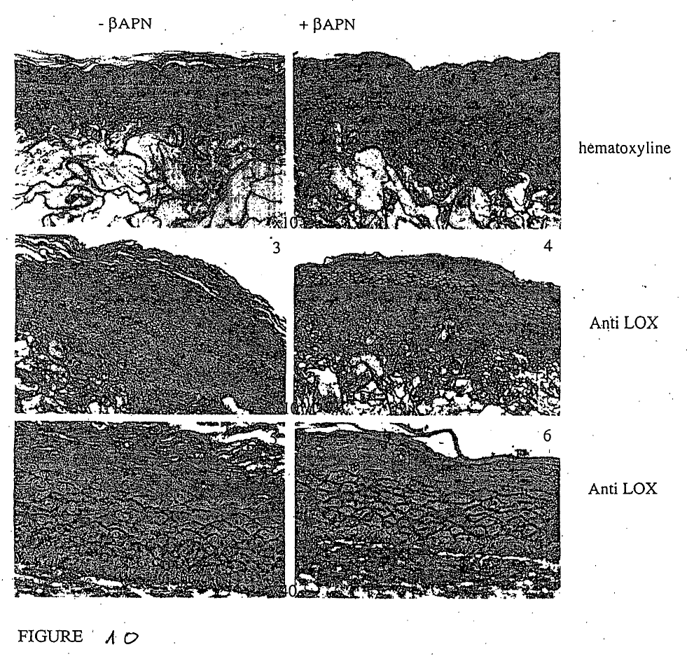

An important effect of βAPN for the grafts would be the possible reduction in the retraction induced by the cross-linking of collagens (FIG. 10).

The surface of the reconstructed skins immersed and treated with βAPN is greater than that of the RSs which are immersed and not treated, suggesting a weakening of the retraction phenomenon. In fact, if the results obtained before the raising to the air-liquid interface are considered, the non-treated control skins show a reduction in surface area of 40% compared to their original state, compared to only 17% for the skins treated with 500 μg/ml of βAPN.

At 35 days of culture, the surface area of the control reconstituted skins or those treated with 50 μg/ml of βAPN is 50% compared to the initial original surface area, while the reconstructed skins treated with 200 and 500 μg/ml of β-APN only have a surface area of 40%. The effect of the βAPN observed on the surface of the reconstructed skins after two weeks of culture at the air-liquid interface is no longer dose-dependent this time. A significantly greater surface area of the RSs treated with 200 or 500 μg/ml of βAPN is noted, compared to that of the control. The RSs treated with 50 μg/ml of βAPN do not show any significant difference in their surface area compared to the non-treated control.

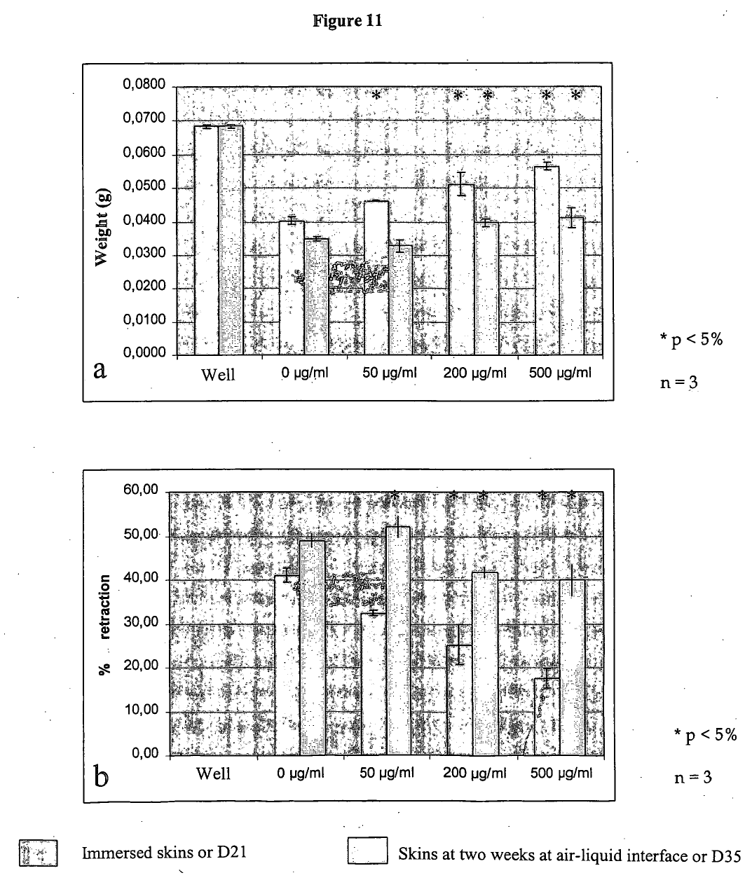

The histology of the RSs (FIG. 11) does not seem to be fundamentally changed by the treatment with βAPN at 50 μg/ml or 200 μg/ml. The treated RSs appear constituted by a dermis having a rich extracellular matrix (ECM), and an epidermis having the basal, spiny, granular layers and stratum corneum. Even if the RSs formed are more fragile and the epidermis is finer, the standard general structure of the epidermis is preserved. The initial colonization of the sponge by the fibroblasts is encouraged by the presence of βAPN, which translates into increased synthesis of ECM.

In the normal skin and in our model of reconstructed skin, the keratinocytes of the basal layer represent the stem cells. The evolution of their concomitant phenotype and their migration towards the external layers of the epidermis will allow the formation of the stratum corneum. This stratum corneum is indispensable, as it ensures the barrier function of the skin. Its formation must however be controlled in order to allow the synthesis of sufficiently large samples of skin. Even if the temporary addition of βAPN seems to slow down the evolution of the initial phenotype of the keratinocytes, the general organization into basal, granular and spiny layers and stratum corneum is maintained.

The immunohistological and ultrastructural analyses reveal significant modifications of the treated RSs. The structuring components of the ECM which are the fibrillar collagens are organized into modified fibres. The molecules of collagen secreted are deposited in the form of fibres with irregular diameters, having bonds between adjacent fibres. We have finally shown by in situ hybridization that the treatment with 50 μg/ml of βAPN translated into a slight increase in the detection of the LOX and LOXL genes, both in the dermis and the epidermis.

Legends to the Figures

FIG. 1: Effect of Lysyl Oxidase Activity on the Dedifferentiation of Chondrocytes after 2 Weeks of Culture in Two Dimensions on Plastic

4 million chondrocytes from the cranial section of the sternum of chicken embryos were inoculated per Petri dish 35 mm in diameter (P35) in DMEM medium with or without 10% serum (replaced by insulin 100 ng/ml) with or without βAPN (50 μg/ml). The culture media with 25 μg/ml of ascorbate are changed every two days. The neosynthesized collagens labelled with Proline 14C for the last 24 hours are isolated after standard pepsination of the conditioned culture media then subjected to SDS PAGE, without reducing conditions, and fluorography.

Observations:

in the presence of serum, which is strongly anabolic, the biochemical dedifferentiation of the chondrocytes (collagen I secreted, stopping of the synthesis of collagen X) is totally inhibited under the action of βAPN which increases the proportion of collagen X specific to these hypertrophic chondrocytes. Similarly, βAPN preserves the hypertrophy and non-spread morphology characteristic of these chondriomes.

in the absence of serum, the chondrocytes adhere to the plastic of the dishes but do not spread. After 15 days of culture, the absence of βAPN only generates the single morphological alteration, but the cell growth overall remains slowed down despite the presence of insulin.

FIG. 2: Effect of Lysyl Oxidase Activity on the Dedifferentiation of Chondrocytes after 3 Days of Culture, in the Presence of Serum, in Two Dimensions on Plastic

The fluorographic study described for FIG. 1 here concerns the pepsinated lawns and culture media

Observations:

Only the presence of βAPN allows the normal expression of collagen X (lower bands of the fluorographs) by the hypertrophic chondrocytes (cranial) identified in the media and the cell lawns. The inhibition of the synthesis of collagen X is known to precede the appearance of collagen I by the chondrocytes during dedifferentiation, which is therefore initiated in the first days of culture while the abnormal collagens have not yet been synthesized.

FIG. 3: Immunodecoration with Anti LOX, LOXL1 Antibodies, of Extracts of Cell Lawns and Culture Medium of Chondrocytes Subjected to SDS PAGE then Transferred onto Nitrocellulose Membrane.

The day before the samples are taken, the cell lawn is washed three times with medium without serum. The medium is then changed for 24 hours with complete medium without serum.

On the 6th or 14th day of culture the media are sampled and precipitated in a standard fashion with TCA 10% final/4° C./30 nm. The centrifugation pellets are rinsed with acetone then solubilized in a 50 μl electrophoresis buffer. The rinsed cell lawns are lysed 2 hours/4° C. (300 μl/P35) in the mixture 8M urea, 0.5% Nonidet, 16 mM disodium phosphate pH 8 and protease inhibitors. The concentration of urea is adjusted to 4M before centrifugation of the suspension. The supernatants are precipitated with TCA then treated as for the corresponding media.

After separation by SDS PAGE and transfer by Western Blot, the protein bands are analyzed by immunodecoration using polyclonal anti-LOX and anti-LOXL1 antibodies from rabbits, then a second anti-rabbit antibody marked with alkaline phosphatase before development with the reagent NBT/BCIP (Roche Diagnostic GmBH, Mannheim, Germany).

Observations:

1) LOX and LOXL1 are expressed by the chondrocytes cultured under the conditions described for the enzymatic activity assay. The Lysyl oxidases accumulated in the lawns after 1 to 2 weeks can be shown, unlike the culture media of only 24 hours.

2) The presence of βAPN does not modify the expression of Lysyl oxidase molecules, only the enzymatic activity is inhibited as Table I shows.

FIG. 4: Identification by Fluorography of the Radio-Labelled Collagens Synthesized by the Chondrocytes of Cotyloid Cartilages in 2D Culture on Plastic. Effects of Hyaluronidase Added to the Enzymatic Digestion Medium of the Cartilages

The 24-hour radio-labellings with the 14C proline were carried out on the 2nd, 7th and 14th day of culture in the presence of serum and βAPN (day 2, day 7 and day 14 respectively). The pepsinated extracts of culture medium and cell lawn were subjected to electrophoresis then fluorography.

The superficial areas (even numbers) and deep areas (odd numbers) of the cotyloid cartilage were separated before being subjected to enzymatic digestion in the presence or absence of hyaluronidase.

Observations:

b) the two sub-populations of chondrocytes, deep and superficial, show a cartilage phenotype on the 2nd day of culture, when they do not synthesize any collagen I, in the cell lawn or in the medium.

c) In the presence of hyaluronidase during the digestion of the cartilage beforehand: the deep area continues not to synthesize collagen I, as on the 7th and on the 14th day of culture. Conversely, the cells of the superficial area dedifferentiate increasingly producing collagen I from the 7th to the 14th day of culture. Neosynthesized collagen I is also present in the cell lawn. Collagen III is produced later and in a small amount on the 14th day in the culture medium, but is only retained in a very small amount in the cell lawn, unlike collagen II.

d) In the absence of hyaluronidase during the digestion of the cartilage beforehand: the two sub-populations of chondrocytes which have just been defined develop in a similar way during the two weeks of culture while dedifferentiating.

FIG. 5: Morphology of the Cotyloid Cartilage Cells in 2D Culture on Plastic.

The observations were carried out on the 7th and 14th days of culture (day 7 and day 14) in the presence of serum and βAPN on the same cultures used for the radio-labellings.

Previous enzymatic digestions of the cartilages in the presence (+) or absence (−) of hyaluronidase.

Observations:

e) digestion in the presence of hyaluronidase (+): on the 7th day of culture (a, e): the cells of the deep area showing a morphology which is characteristic of the chondrocytes, as can be observed in situ. By contrast, the cells of the superficial area, of the fibroblast type, have numerous fine extensions. These differences are maintained during the successive multiplications at least until the 14th day of culture (c, g).

f) digestion in the absence of hyaluronidase (−): the cells are included in a more abundant extracellular matrix where the morphological differences between the two sub-types of cells are less clear (b, f, d and h).

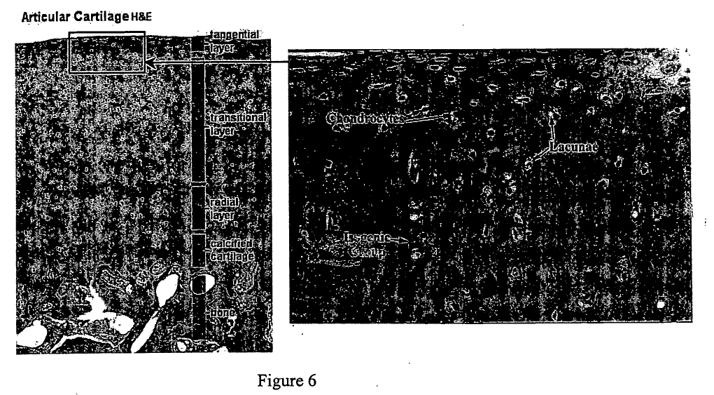

FIG. 6: Structure of Normal Human Cartilage

FIG. 7: Normal Human Chondrocytes Cultured for 1 Month in 3D Under Stirring in the Presence of βAPN

3 D culture in sponges of collagen-GAG-chitosan in the presence of serum and βAPN of normal human chondrocytes. The chondrocytes were previously multiplied, and dedifferentiated, in 2D culture on plastic.

The histological analysis shows at the surface the presence of a dense thick area with a histological structure very close to that of normal human cartilage (FIG. 6). The chondrocytes are surrounded by a large extracellular matrix which they have neosynthesized and are accommodated in small voids. Deeper, a colonization of the sponge with a very weak matrix synthesis is observed.

FIG. 8: Normal Human Chondrocytes Cultured for 1 month in 3D Under Stirring Without βAPN

The same sponges of collagen-GAG-chitosan treated here without βAPN have at the surface a dense and not very thick area localized on a limited surface of the sponge. Deeper, the colonization and the synthesis of the matrix are no different to the sponges treated with βAPN.

In conclusion the treatment with βAPN seems to favour the development of a structure close to that of normal human cartilage at the surface.

FIG. 9: Reversibility of the Effects of βAPN on the Bridgings of the Chains of Collagen IX Produced in the Cell Lawn of Hypertrophic Chondrocytes of Chicken Embryos. Fluorographic Study.

Chondrocytes of the cranial section of the sternum were inoculated at a high concentration (5 million cells per P35 Petri dish) then subjected to 24 hours of radio-labelling with proline 14C on the 7th, 14th and 24th day of culture (respectively day 7, day 14 and day 21).

βAPN was present (+) or absent. (−) for the entire duration of the culture, or present only during the first week ((+)).

Observations:

The presence of two main bands of collagen IX varies as a function of the presence or absence of βAPN during the entire culture: band A is connected with the absence of βAPN, while band B is observed essentially in the presence of βAPN. The presence of βAPN limited to only the first week gives an electrophoretic profile after 2 and 3 weeks of culture which is characteristic of a culture without βAPN.

In all the cases, the synthesis of collagen X increases with the age of the culture, but the synthesis of collagen I is slowed down by the high density of initial inoculation (5 million cells/P35 instead of 1.5).

Conclusions:

The effect of βAPN on the collagen IX bridgings is shown; it precedes that on the dedifferentiation characterized by the synthesis of collagen I. These results show:

b) the reversibility of the effects of βAPN.

c) the effectiveness of the elimination of βAPN from the cell culture after repeated washings of the cell lawns then use of a culture medium without βAPN.

FIG. 10: Effect of β-APN on the Retraction of Reconstructed Skins:

β-APN brings about a significant dose-dependent reduction of the retraction compared to the control, when the skins are still in immersed culture (in grey). After two weeks at the air-liquid interface (in white), the retraction is still reduced compared to the control but only for the concentrations 200 and 500 μg/ml.

FIG. 11: Immunohistochemical Analysis of the Reconstructed Skin with and Without βAPN

1 and 2: staining of control haematoxylin without 1st antibody.

3 to 6: immunodetection of the LOX by a 2nd antibody marked with peroxidase in the absence (1, 3, 5) or presence (2, 4, 6) of βAPN

The dark brown stains mark the presence of the antigen LOX and are absent from the Control fractions.

After 35 days of formation, the reconstructed skin is constituted by a dermis deposited on the collagen-glycosaminoglycan-chitosan sponge, and by an epidermis formed by the keratinocytes.

At the level of the dermis, the treatment with βAPN allows a larger colonization of the sponge by the fibroblasts, which translates into an increased synthesis of extracellular matrix (1 and 2). The expression of LOX appears more intense.

At the level of the epidermis, the general stratification is recovered, but the

TABLE I
Lysyl oxidase activities noted in the cell lawns of chondrocytes in culture
on plastic. Two populations of chondrocytes from the sternum of chicken
embryos (hypertrophic in the cranial section, synthesizing collagen X,
and not hypertrophic in the caudal section)
Inoculation: 4 million cells/35 mm Petri dish (P35).
Culture + 25 μg/ml ascorbate, + 1 mM pyruvate, ± 10% FCS, ± 1 mM
cys. 24 hours before the collection of the medium without serum (1.5 ml)
the cell lawn is washed with the medium without serum + cys. Aliquots of
the culture medium after 24 hours are concentrated 10 times then subjected
to the Lysyl oxidase activity assay with different batches of tritiated
elastin substrate.

| | dpm − βAPN | dpm + βAPN | activity/P 35 |
|---|---|---|---|
| Experiment 1 | | | |
| (substrate no. 1) | | | |
| (culture − FCS, + cys) | | | |
| Day 3 | | | |
| caudal chondrocytes | 930 | 674 | 1925 |
| (15% medium P35) | 1002 | 718 | |
| Experiment 2 | | | |
| (substrate no. 2) | | | |
| (culture + FCS, − cys) | | | |
| Day 7 | | | |
| caudal chondrocytes | 8158 | 5296 | 10232 |
| (25% medium P35) | 8464 | 6210 | |
| cranial chondrocytes | 10932 | 5800 | 4553 |
| (100% medium P35) | 10510 | 6536 | |
| standard purified aortic | 8544 | 4340 | / |
| LO | 8810 | 4762 | |
| Day 13 | | | |
| caudal chondrocytes | 5016 | 4344 | 861 |
| (75% medium P35) | 5322 | 4702 | |
| cranial chondrocytes | 5038 | 4580 | 840 |
| (100% medium P35) | 5840 | 4618 | |

### CONCLUSIONS

Lysyl oxidase activities (inhibited by βAPN and assayed by the production of tritiated water) are secreted in the culture media. The specific activity recorded in the media is stronger on the 7th day of culture than on the 13th day. The extracellular matrix being richer in natural collagenic substrates of the Lysyl oxidase activity on the 13th day of culture, it would thus retain the enzyme in the cell lawn.

1. A method for inhibiting the dedifferentiation of cells in culture in vitro, comprising contacting said cells with a direct or indirect lysyl oxidase (LO) to inhibit the dedifferentiation of cells in culture in vitro to maitain the phenotype of said cells and for the whole of a culture period.

2. The method according to claim 1, for the preparation of differentiated cells the phenotype of which is identical, or for the preparation of a cellular matrix constituted by differentiated cells preserving the same phenotype and cultured in the presence of said inhibitors, and of the extracellular medium secreted by said cells and binding the latter in said matrix, said cellular matrix being capable of being used as tissues or tissue implants.

3. The method according to claim 1, wherein said inhibitors are direct LO inhibitors chosen from:
A) primary amines reacting with the carbonyl group of the active site of the LOs, and more particularly those which produce, after binding with the carbonyl, a product stabilized by resonance, such as the following primary amines:
ethylenediamine,
hydrazine, phenylhydrazine, and their derivatives, semicarbazide, and urea derivatives,
aminonitriles, such as β-aminopropionitrile (β-APN), or 2-nitroethylamine,
unsaturated or saturated haloamines, such as 2-bromo-ethylamine, 2-chloroethylamine, 2-trifluoroethylamine, 3-bromopropylamine, 8-halobenzylamines, and unsaturated halogen compounds,
selenohomocysteine lactone,
B) copper chelating agents, penetrating or not penetrating the cells in culture,
C) anti-LO blocking antibodies directed against the active site of the LO.

4. The method according to claim 1, wherein said inhibitors are indirect LO inhibitors chosen from:
A) the compounds blocking the aldehyde derivatives originating from the oxidative deamination of the lysyl and hydroxylysyl residues by the LOs, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphinate, 2-acetamidoethyl-2-acetamidoethanethiol sulphanate, sodium-4-mercaptobutanesulphinate trihydrate.
B) the compounds inhibiting the biosynthesis of LOs such as the antisenses.

5. The method according to claim 4, wherein said, of LO inhibitors are chosen from β-APN and/or D-penicillamine.

6. The method according to claim 1 for the implementation of methods of in vitro culture of all cells of human or animal origin, in which said cells are maintained in a phenotype with constant differentiation, and are chosen in particular from the chondrocytes of the cartilages, cornea cells, skin cells (such as the dermal fibroblasts, and epidermal keratinocytes), endothelial cells of the vessels, bone osteoblasts, hepatocytes, renal cells, muscle cells, stem or pluripotential cells.

7. The method according to claim 1, for the implementation of methods of in vitro culture of cells, in order to obtain cells capable of being used in cell therapy, or in experimental pharmacology, in particular in the screening of medicaments.

8. The method according to claim 1, for the implementation of methods of in vitro culture of cells in order to obtain tissues, such as skin or cartilage tissues, said tissues being able of being used as grafts or implants in tissue therapy, or in experimental pharmacology, in particular in the screening of medicaments.

9. Method of in vitro culture of cells, during which the phenotype of said cells is maintained at a stage identical to that in which said cells were found initially during their culture, said method comprising, culturing said cells in an appropriate medium containing one or more lysyl oxidase (LO) inhibitors, wherein said inhibitors are direct or indirect LO inhibitors.

10. Method for preparing cells, or cell implants, characterized in that it comprises:
the implementation of an in vitro cell culture method according to claim 9,
if appropriate, one or more stages of washing the cells in culture using an appropriate buffer solution, in order to completely or partially eliminate the LO inhibitor or inhibitors used,
if appropriate, a stage of enzymatic digestion of the extracellular material capable of being formed, using appropriate enzymes,
if appropriate, a stage of recovery of the cells cultured.

11. Method for preparing in vitro a cell matrix, capable of being used as tissues or tissue implants, constituted by differentiated cells with identical phenotype, characterized in that it comprises:
culturing cells according to claim 9, until formation of a cell matrix as defined above, which is sufficient to constitute a tissue stroma,
if appropriate, one or more stages of washing of the cell matrix in culture using an appropriate buffer solution, in order to completely or partially eliminate the LO inhibitor or inhibitors used,
if appropriate, a stage of recovery of the cell matrix as defined above.

12. The method according to claim 11, wherein said method is carried out from chondrocytes, or for the preparation of cutaneous substitutes when said method is carried out from fibroblasts and/or keratinocytes.

13. Method for screening molecules of pharmacological interest, in particular medicaments, characterized in that it comprises:
culturing cells according to claim 9, if appropriate, until the formation of a cell matrix which is sufficient to constitute a tissue stroma,
if appropriate, one or more stages of washing the tissues in culture using an appropriate buffer solution, in order to completely or partially eliminate the LO inhibitor or inhibitors used,
a stage of placing the molecule tested in the presence of the cells or tissues obtained during the preceding stages,
detecting any effect of said molecule on said cells or tissues.

14. Cells or cell implants, or tissues or tissue implants produced by culturing said cells or cell implants according to claim 9.

15. Cell implants according to claim 14, corresponding to implants of suspensions of chondrocytes, the initial cartilage-type phenotype of which, was maintained during the preliminary phase of cell multiplication in vitro.

16. Cells or cell implants, or tissues or tissue implants produced by culturing said cells or cell implants according to claim 15.

17. Tissue implants according to claim 14, corresponding to cartilage implants comprising an extracellular matrix which is essentially free from collagens which are normally absent from healthy cartilage (namely collagens I and III), and comprising all of the collagens specific to cartilage (namely the collagens II, IX, XI, and if appropriate X).

18. Cartilage implants according to claim 17, wherein said cartilage implants are produced by a process according to claim 11.

19. Tissue implants according to claim 14, wherein said implants are of reconstituted skin, or cutaneous substitute.
